(19) United States
(12) Patent Application Publication
(10) Pub. No.: US 2024/0407879 A1
(43) Pub. Date: Dec. 12, 2024

(54) FIXATION ELEMENT, REPROCESSING TRAY, AND METHOD OF REPROCESSING A MEDICAL DEVICE

(71) Applicant: OLYMPUS Winter & Ibe GmbH, Hamburg (DE)

(72) Inventors: Michael Wolter, Hamburg (DE); Uwe Schoeler, Hamburg (DE)

(73) Assignee: OLYMPUS Winter & Ibe GmbH, Hamburg (DE)

(21) Appl. No.: 18/736,718

(22) Filed: Jun. 7, 2024

Related U.S. Application Data
(60) Provisional application No. 63/471,682, filed on Jun. 7, 2023.

(51) Int. Cl. A61B 50/33; A61L 2/26

(52) U.S. Cl. CPC A61B 50/33; A61L 2/26; A61L 2202/24

(57) ABSTRACT

Disclosed herein is a fixation element for holding a medical instrument during a reprocessing procedure, wherein the fixation element is configured to undergo a shape change during the reprocessing procedure. Also disclosed are a reprocessing tray and a method of reprocessing a medical instrument.

### CROSS-REFERENCE TO RELATED APPLICATION

The present application is based upon and claims the benefit of priority from U.S. Provisional Application No. 63/471,682 filed on Jun. 7, 2023, the entire contents of which is incorporated herein by reference.

### BACKGROUND

### Field

The present disclosure relates to reprocessing medical instruments. More specifically, the present disclosure relates to fixation elements for holding a medical instrument during reprocessing.

### Prior Art

Reusable medical instruments need to be carefully reprocessed after each use, before they can used on another patient. Reprocessing usually involves several steps, including manual precleaning and automated disinfection or sterilization. The automated disinfection or sterilization is usually performed in sophisticated reprocessing machines or autoclaves, where the medical instruments are treated with heated fluids, such as liquids, gases, steam, plasma, and/or chemicals.

During automated disinfection of sterilization, medical instruments undergoing reprocessing are usually held in reprocessing trays, which are also used for transporting the medical instruments to and from the reprocessing machine or autoclave.

In order to enable effective reprocessing, the medical instruments undergoing reprocessing must be held in a suitable position during treatment. Therefore, reprocessing trays are equipped with fixation elements, which hold medical instruments in the desired position. The desired position may be a position which avoids direct contact between multiple medical instruments in the reprocessing tray, or between different sections of a deformable medical instrument, such as a flexible endoscope.

Known fixation elements usually comprise elastic material shaped to conform with the medical instrument to be held. The shape of known fixation elements needs to be designed to strike a suitable balance between firmly holding the instrument and allowing reprocessing fluids or steam to contact as much surface area of the medical instrument as possible. Due to this design dilemma, fixation elements may either cover too much surface area of a medical instrument, or allow too much movement of a medical instrument, so that the medical instrument may dislodge from the fixation elements during transport.

It would be desirable to provide an improved fixation element for holding medical instruments during reprocessing.

### SUMMARY

The present disclosure provides a fixation element for a medical instrument, wherein the fixation element is configured to undergo a shape change during the reprocessing procedure. Through the shape change, the fixation element may firmly hold the medical instrument during transport to or from a reprocessing facility, such as a reprocessing machine or an autoclave, and may still allow reprocessing fluids or steam to contact a sufficient portion of the medical instrument's surface to provide effective disinfection or sterilization.

The fixation element may be configured to undergo the shape change in response to a change in temperature. Automated reprocessing of medical instruments usually involves subjecting the medical instrument to elevated temperatures. These elevated temperatures can thus be used to initiate the shape change.

The fixation element may be configured to change between a first shape and a second shape, the first shape being configured to firmly hold the medical instrument such that unwanted movement or dislodging of the medical instrument during transport is avoided, the second shape being configured to loosely hold the medical instrument while allowing reprocessing fluids or steam to contact the surface of the medical instrument. The second shape may be selected to provide a reduced contact surface between the fixation element and the medical instrument.

The fixation element may be configured to automatically change from the first shape to the second shape when the temperature of the fixation element rises above a predetermined transition temperature. The fixation element may be configured to automatically change from the second shape to the first shape what the temperature of the fixation element falls below the predetermined transition temperature. The fixation element may be configured to be deformed from the second shape to the first shape by applying an external force while the temperature is below the predetermined transition temperature.

The fixation element may comprise one or more of a bimetal element, a shape memory alloy (SMA) element, and a shape memory polymer (SMP) element. The fixation element may further comprise an elastic material covering the at least one of a bimetal element, an SMA element, and an SNIP element. The fixation element may further comprise a bias element. The bias element may be configured to create an elastic force urging the fixation element into the first shape or into the second shape.

The fixation element may comprise a first contact feature for contacting the medical instrument when the fixation element is in the first shape, and a second contact feature for contacting the medical instrument when fixation element is the second shape. The first contact feature may have a larger contact surface than the second contact feature.

The present disclosure further provides a reprocessing tray for medical instruments, comprising a tray body and one or more fixation elements according to the above description. The reprocessing tray may additionally include one or more fixation elements according to the prior art, not being capable of undergoing a shape change. Fixation elements according to the present disclosure may be provided predominantly in positions corresponding to areas of a medical instrument which are difficult to reprocess, like complex surface geometries, transitions between metal and plastic parts, or the like.

The present disclosure further provides a method of reprocessing a medical instrument comprising: providing a reprocessing tray as described above; inserting the medical instrument in the reprocessing tray, so that the medical instrument is held by one or more fixation elements being in the first shape; subjecting the reprocessing tray, the one or more fixation elements, and the medical instrument to an elevated temperature, so that the one or more fixation elements transition from the first shape the second shape; and reprocessing the medical instrument while the one or more fixation elements are in the second shape. The reprocessing tray may be provided with the one or more fixation elements being in the second shape, and the method may further comprise, before or after insertion of the medical instrument, deforming the one or more fixation elements from the second shape to the first shape by applying an external force.

### BRIEF DESCRIPTION OF THE DRAWINGS

In the following, some exemplary embodiments of the disclosure are explained at hand of drawings. It is to be understood that the drawings and related description are only provided for better understanding of the disclosure, without the intent to be technically limiting. The scope of protection is defined by the appended claims.

In the drawings:

FIG. 1 illustrates a schematic overview of a reprocessing process for a medical device;

FIGS. 2a and 2b illustrate a fixation element in first and second shapes;

FIG. 3 illustrates the fixation element of FIG. 2a in a longitudinal sectional view;

FIG. 4 illustrates the fixation element of FIG. 2b in a transverse sectional view; and

FIGS. 5a-5c illustrate exemplary methods of reprocessing a medical instrument.

### DETAILED DESCRIPTION

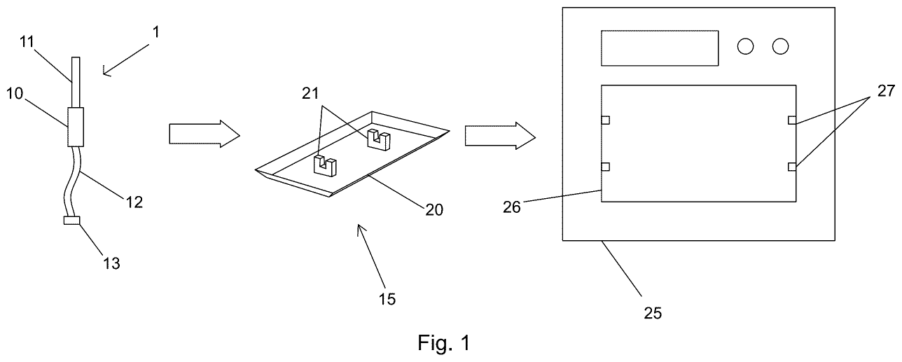

FIG. 1 shows a schematic overview of a reprocessing process for a medical device 1. In the shown example, the medical device 1 is a surgical video endoscope having a main body 10, an elongate shaft 11, a cable 12, and a plug 13.

After the medical device 1 has been used with a patient in a surgical procedure, it needs to be carefully reprocessed before reuse on a further patient, so that cross-contamination with pathogens can securely be prevented.

Reprocessing usually involves manual precleaning, wherein body fluids and other residues are removed from the surface and any internal channels of the medical instrument. Precleaning can be done using brushes, wipes, and low temperature detergents. The precleaning step not shown in FIG. 1 as it is not essential for the understanding of the present disclosure.

After precleaning, the medical device 1 is placed in a reprocessing tray 15. The reprocessing tray 15 comprises a tray body 20 and a number of fixation elements 21. The tray body 20 may comprise a thermoplastic or thermosetting polymer, a sheet metal, a wire mesh, or a plastic mesh. In case of a polymer or sheet metal tray body, openings (not shown) may be provided in the tray body to enable diffusion of steam during reprocessing in an autoclave. Wire or plastic mesh trays are more commonly used for reprocessing in endoscope reprocessing machined using liquid reprocessing agents.

The fixation elements 21 are configured to secure the medical instrument 1 in the reprocessing tray 15. Usually, the fixation elements 21 comprise bodies of an elastic polymer like silicone, which is sufficiently soft to prevent any scratching or cutting of the surface of the medical instrument 1. The fixation elements 21 must hold the medical instrument 1 strong enough to prevent dislodging of the medical instrument 1.

The reprocessing tray 15 with the medical instrument 1 placed therein is then transported to an automatic reprocessing device 25, such as an endoscope reprocessing machine or an autoclave.

The reprocessing device 25 has an internal reprocessing chamber 26, into which the reprocessing tray 15 can be inserted through a door (not shown). The reprocessing chamber 26 may be equipped with rails 27 for holding one or more the reprocessing trays 15.

In the processing chamber, the medical instrument 1 is subjected to treatment with chemical detergents, disinfectants, water, gas, plasma, and/or steam at elevated temperatures. For effective reprocessing, the substances used for reprocessing must be able to reach all places of a surface of the medical instrument 1. However, the fixation elements 21 may block access to the surface in places where the fixation elements 21 touch the medical instrument 1.

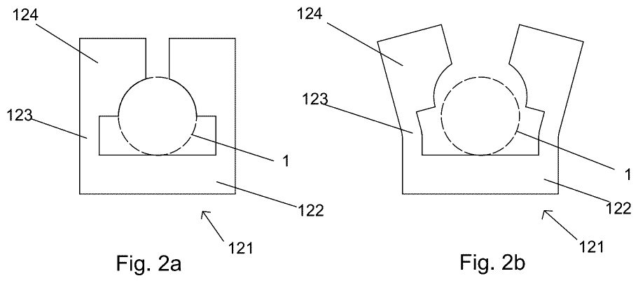

FIGS. 2a and 2b show a fixation element 121, which may be used in the reprocessing tray 15. The fixation element 121 is configured to hold a medical instrument 1 (shown in dashed lines). The fixation element comprises a base 122, arms 123 extending from the base 122, and contact pads 124 attached to the arms 123.

FIG. 2a shows the fixation element 121 in a first shape, where the arms 123 are in a straight configuration and the contact pads 124 are bent inwards for contacting the medical instrument 1. The medical instrument 1 is therefore securely held between the base 122, such as during transport prior to reprocessing, on which the medical instrument 1 rests due to gravity, and the contact pads 124.

FIG. 2b shows the fixation element 121 in a second shape, where the arms 123 are bent away from each other by a small angle. Consequently, the contact pads 124 are bent outwardly to release the medical instrument 1 when the medical instrument is in the reprocessing device 25 and undergoing reprocessing, so that the medical instrument 1 only rests on the base 122.

The shape change of the fixation element 121 is initiated by a temperature change occurring while the medical instrument 1 undergoes reprocessing in the reprocessing device 25.

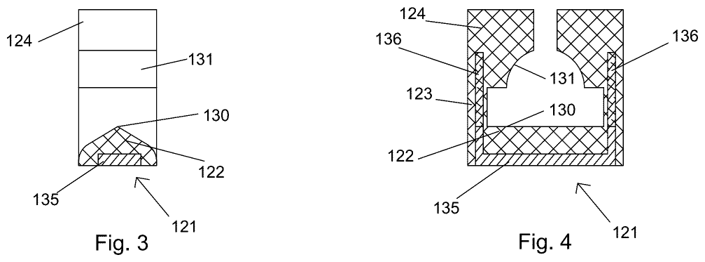

FIG. 3 shows a longitudinal sectional view of the fixation element 121 shown in FIG. 2a. FIG. 4 shows a transverse sectional view of the fixation element 121. It can be seen that the base 122 has a prismatic cross-section ending in an upper tip or ridge 130, which serves as lower contact feature for contacting the medical instrument (not shown in FIG. 3) with a very small contact surface, so that a surface of the medical instrument is accessible by reprocessing liquids or steam. The contact pad 124 comprises an upper contact feature 131 in form of a rounded surface. The upper contact feature comprises a larger contact surface than the lower contact surface, and provides for securely holding the medical instrument. Instead of the contact features shown, other contact features may be provided, which may be in the shape of domes, bumps, cones, or the like.

The fixation element 121 mainly comprises a soft elastic material in order to avoid damage to the medical instrument. The material may be silicone (polysiloxane). A rigid core 135 in the base 122 of the fixation element 121 increases the overall stability of the fixation element 121 and may be used for fixing the fixation element 121 to a reprocessing tray, e.g. by screws (not shown). The core 135 may comprise metallic material, like medical grade steel. In other embodiments, the core may also comprise silicone.

From FIG. 4 it can be seen that the core 135 extends in the direction of the arms 123 on both sides of the base, where it is connected to shape changing elements 136. The shape changing elements 136 are configured to change their shape from a straight shape, as shown in FIG. 4, to a bent shape (not shown), so that the fixation element 121 assumes the overall shape as shown in FIG. 2b, when a temperature of the fixation element 121 rises above a predetermined transition temperature.

The shape changing elements 136 may comprise bimetal elements. Bimetal elements comprise layered structures of metals with different thermal expansion coefficients. In an example, the shape changing elements 136 may comprise a strip of metal with high thermal expansion coefficient, like Zinc, on the side facing the base, and a strip of metal or metal alloy with a low thermal expansion coefficient, like an alloy comprising 64% of iron and 36% of nickel, known as Invar, on the side facing away from the base. The respective strips may have the same length at room temperature. When the temperature increases, the Zinc strip will lengthen more than the Invar strips, so that the shape changing elements 136 bend to the outside. After the temperature returns to a room temperature level, the shape changing elements will revert to their straight shape again.

In other embodiments, the shape changing elements may comprise shape memory alloys (SMA) or shape memory polymers (SMP). SMAs and SMPs generally comprise a “home” shape, which is stable. By applying an external force, SMAs and SMPs may be deformed into a different shape, which is quasi-stable while the shape changing element is below a transition temperature. When the shape changing element is heated above the transition temperature, the element autonomously or automatically reverts to its “home” shape, which it will maintain irrespective of the temperature. In embodiments with shape change elements 136 from SMA or SMP, the shape change elements 136 may be provided with their “home” shape being bent outwardly, so that the fixation element 121 assumes the shape depicted in FIG. 2b. By applying an inwardly directed force, e.g. by pushing the contact pads 124 inwardly by hand, the shape change elements 136 can be deformed into straight shape, so that the fixation element 121 assumes the shape depicted in FIG. 2a. When the fixation element 121 is heated above the transition temperature in the reprocessing device 20, the shape changing elements 136 revert to their bent “home” shape, so that the fixation element assumes the shape according to FIG. 2b again.

In some embodiments, the force required to deform an SMA or SMP from the “home” shape into a different shape may be provided by elastic components of the fixation element. For example, a silicone body of the fixation element may provide sufficient elastic force to deform an SMA or SMP element from a straight “home” shape, which may be the shape depicted in FIG. 4, into a bent shape. Such deformation may further be supported by an additional bias element (a biasing material), such as a leaf spring, which may be integrated in the shape change elements 136. When the shape change elements 136 are heated above the transition temperature, the reforming force of the shape change element may be higher than the deforming force of the silicone body or the bias element, so that the shape change elements 136 return into the straight “home” shape. After the temperature of the shape change elements 136 drop below the transition temperature, the elastic forces may again prevail, so that the shape change elements 136 are again elastically deformed into the bent shape.

The transition temperature of the shape changing elements 136 can be selected according to the temperatures applied during reprocessing. After selection of a suitable transition temperature, suitable SMAs or SMPs can be chosen from available materials. Suitable elastic deforming forces and reforming forces can be achieved by material selection and size determination of respective elements. In some embodiments, the body of the fixation element may comprise silicone with a Shore hardness between 30 or 50.

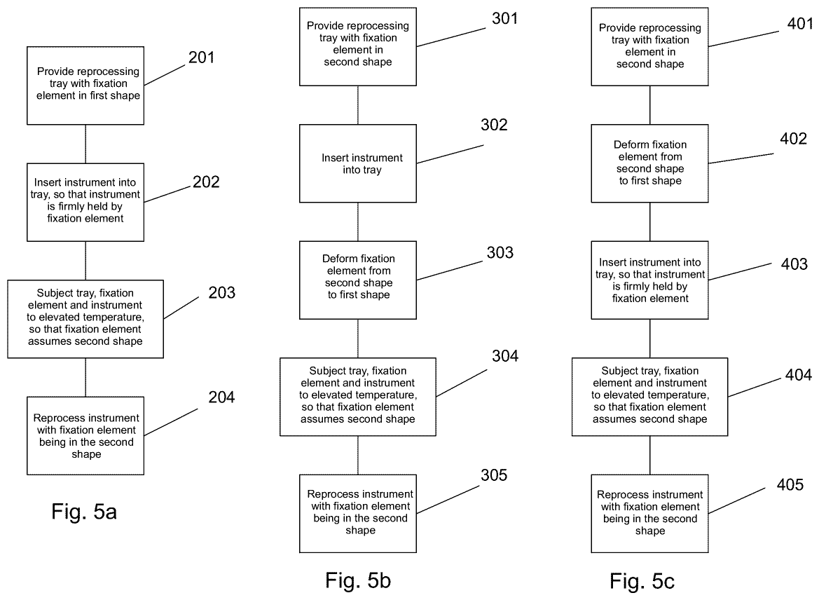

FIGS. 5a to 5c show some exemplary methods of reprocessing a medical instrument using the fixation elements and reprocessing trays described herein.

As shown in FIG. 5a, in a first exemplary method, in step 201, a reprocessing tray is provided with one (or more) fixation elements in a first shape. In step 202, a medical instrument is inserted into the tray, so that the instrument is firmly held by the one or more fixation elements. In step 203, the tray, the fixation element, and the instrument are subjected to an elevated temperature. Through the elevated temperature, the one or more fixation elements assume the second shape. In step 204, the medical instrument is then reprocessed with the one or more fixation elements in the second shape. After the reprocessing, the fixation elements may automatically return into the first shape by elastic force.

FIG. 5b shows a further method, in which the reprocessing tray is provided in step 301 with the one or more fixation elements being in the second shape. The fixation elements may comprise shape changing elements comprising SMAs or SMPs, which do not return to the first shape automatically. In step 302, the instrument is inserted into the reprocessing tray, while the one or more fixation elements are still in the second shape, before the one or more fixation elements are deformed into the first shape in step 303. The final steps 304 and 305 are identical with steps 203 and 204 of the method show in FIG. 5a.

FIG. 5c shows a further exemplary method, in which again the reprocessing tray is provided in step 401 with the one or more fixation elements being in the second shape. In step 402, the one or more fixation elements are deformed into the first shape, before the medical instrument is inserted into the reprocessing tray in step 403. Steps 404 and 405 are again identical to steps 203 and 204 of the method shown in FIG. 5a.

While there has been shown and described what is considered to be embodiments of the invention, it will, of course, be understood that various modifications and changes in form or detail could readily be made without departing from the spirit of the invention. It is therefore intended that the invention be not limited to the exact forms described and illustrated, but should be constructed to cover all modifications that can fall within the scope of the appended claims.

What is claimed is:

1. A fixation element for holding a medical instrument during a reprocessing procedure, wherein the fixation element is configured to undergo a shape change during the reprocessing procedure.

2. The fixation element of claim 1, wherein the fixation element is configured to undergo the shape change in response to a change in temperature.

3. The fixation element of claim 2, wherein the fixation element is configured to change between a first shape and a second shape, the first shape being configured to firmly hold the medical instrument, the second shape being configured to loosely hold the medical instrument.

4. The fixation element of claim 3, wherein the second shape is selected to provide a reduced contact surface between the fixation element and the medical instrument.

5. The fixation element of claim 3, wherein the fixation element is configured to automatically change from the first shape to the second shape when the temperature of the fixation element rises above a predetermined transition temperature.

6. The fixation element of claim 5, wherein the fixation element is configured to automatically change from the second shape to the first shape when the temperature of the fixation element falls below the predetermined transition temperature.

7. The fixation element of claim 5, wherein the fixation element is configured to maintain the second shape when the temperature of the fixation element falls below the predetermined temperature.

8. The fixation element of claim 7, wherein the fixation element is configured to be deformed from the second shape to the first shape by applying an external force while the temperature is below the predetermined transition temperature.

9. The fixation element of claim 8, wherein the fixation element comprises one or more of a bimetal element, a shape memory alloy (SMA) element, and a shape memory polymer (SMP) element.

10. The fixation element of claim 9, further comprising an elastic material covering the at least one of the bimetal element, the SMA element, and the SMP element.

11. The fixation element of claim 3, further comprising a biasing material for biasing the fixation element into one of the first shape or the second shape.

12. The fixation element of claim 3, wherein the fixation element comprises a first contact surface for contacting the medical instrument when the fixation element is in the first shape, and a second contact surface for contacting the medical instrument when the fixation element is in the second shape.

13. The fixation element of claim 12, wherein the first contact surface is larger than the second contact surface.

14. A reprocessing tray for medical instruments, comprising a tray body and one or more fixation elements according to claim 1.

15. A method of reprocessing a medical instrument, comprising:
providing a reprocessing tray according to claim 12;
inserting the medical instrument in the reprocessing tray, so that the medical instrument is held by the one or more fixation elements being in the first shape;
subjecting the reprocessing tray, the one or more fixation elements, and the medical instrument to an elevated temperature, such that the one or more fixation elements transition from the first shape to the second shape; and
reprocessing the medical instrument while the one or more fixation elements are in the second shape.

16. The method of claim 15, wherein the reprocessing tray is provided with the one or more fixation elements being in the second shape, the method further comprising:
before or after insertion of the medical instrument, deforming the one or more fixation elements from the second shape to the first shape by applying an external force.
